(19) United States
(12) Patent Application Publication
(10) Pub. No.: US 2026/0216435 A1
(43) Pub. Date: Jul. 30, 2026

(54) MEDICAMENT DELIVERY DEVICE AND MEDICAMENT DELIVERY ASSEMBLY

(71) Applicant: SHL Medical AG, Zug (CH)

(72) Inventors: Joakim Gunnar Lindholm, Nacka Strand (SE); Anders Boström, Nacka Strand (SE); Oscar Henrik Alexandersson

(21) Appl. No.: 19/146,391

(22) PCT Filed: Dec. 13, 2023
(86) PCT No.: PCT/EP2023/085545

§ 371 Date: Jul. 8, 2025

(30) Foreign Application Priority Data
Jan. 17, 2023 (EP) 23152093.3

(51) Int. Cl. A61M 5/20; A61M 5/315

(52) U.S. Cl. CPC A61M 5/2033; A61M 5/3157; A61M 2205/581; A61M 2205/582

(57) ABSTRACT

The present disclosure provides a medicament delivery device for accommodating a medicament container, for example a syringe, is provided. The medicament delivery comprises a drive mechanism equipped to act on the medicament container for expelling a medicament therefrom. The drive mechanism may for example comprise a drive spring. For providing a feedback signal, the medicament delivery device further comprises a feedback spring in a pre-tensioned state and a feedback spring release mechanism for releasing the feedback spring from the pre-tensioned state when the medicament has been expelled. Release of the feedback spring causes the feedback signal, for example by the feedback spring tapping against a distal end cap portion.

### TECHNICAL FIELD

The invention is in the field of medicament delivery devices. More in particular, it relates to an automatic medicament delivery device providing audible feedback to a patient who used it. It further relates to an assembly comprising such a device, pre-assembled with a medicament container, for example a syringe.

### BACKGROUND

Medicament delivery devices for automatic delivery of a medicament by self-administration are well-known. Especially, they may be equipped to accommodate a syringe as a medicament container. Often, the medicament delivery device and the medicament container are pre-assembled to constitute a medicament delivery assembly for self-administration.

Such medicament delivery devices and medicament delivery assemblies should generally be safe to use and easy to handle, and they should ensure delivery of the designated dose of the medicament. In view of this, it may be important that the patient receives a feedback signal after delivery of the medicament.

WO 2011/123024 A1 discloses a medicament delivery device for a syringe, wherein the medicament delivery device provides an audible tactile or visible confirmation to a patient when an injection has been made. To this end, the medicament delivery device comprises a signal generating member being an elongated U-shaped bracket. The U-shaped bracket is allowed to be displaced towards distally as soon as the plunger rod that displaces the plunger of the syringe is in the end position. A drive spring, which displaces the plunger rod during delivery, then acts to move the U-shaped bracket towards distally until it taps against the inner surface of the housing, resulting in the desired feedback signal. This solution has the disadvantages that the U-shaped bracket is an additional component and that the energy needed for the feedback signal stems from the drive spring that also needs to move the plunger of the syringe.

### SUMMARY

Accordingly, it is an object of the present invention to provide a medicament delivery device that overcomes at least some of the drawbacks of the prior art. Especially, the medicament delivery device should be advantageous in terms of reliability.

This object is achieved by the invention as defined in the appended claims, to which reference should now be made.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which during use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which during use of the medicament delivery device is/are located closest to the dose delivery site.

Further, the terms “longitudinal”, “longitudinally”, “axially” and “axial” refer to a direction extending from the proximal end to the distal end and along the device or components thereof, typically in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a longitudinal axis extending in the direction of the longest extension of the device and/or component. Similarly, “radial” or “radially” refer to a direction extending radially relative to the axis, and “rotation”, “rotational” and “rotationally” refer to rotation relative to the axis.

When a component is said to move proximally, distally, axially in a proximal direction, axially in a distal direction or equivalent terms, the movement is relative to the housing of the injection device, unless mentioned otherwise.

According to an aspect of the present invention, a medicament delivery device for accommodating a medicament container is provided. The medicament delivery device comprises a drive mechanism equipped to act on the medicament container for expelling a medicament therefrom. If the medicament container is a syringe, the drive mechanism may for example drive the syringe's plunger to expel the medicament via the syringe's needle.

For providing the feedback signal, the medicament delivery device comprises a feedback spring in a pre-tensioned state, and a feedback spring release mechanism for releasing the feedback spring from the pre-tensioned state when the medicament has been expelled. Release of the feedback spring causes the feedback signal. The feedback spring is an axial helical spring.

In contrast to the prior art, therefore, the energy for the feedback signal does not stem from the drive mechanism itself but from a dedicated feedback spring, which is only released from the pre-tensioned state when the medicament has been expelled. Thich means that all energy available to the drive mechanism can be used for expelling the medicament. Also, the stored energy in the feedback spring remains entirely stored until the medicament has been expelled, and all energy stored in the feedback spring can be used for the generation of the feedback signal. The feedback spring therefore does not need to store a large amount of energy and can thus be a relatively inexpensive element.

The medicament delivery device may comprise a housing and a drive member movable relative to the housing by the drive mechanism. For example, the drive mechanism may comprise a drive biasing member. The drive biasing member is pre-loaded to release the energy needed for driving the drive member from an initial position to an end position and for thereby delivering the medicament. Especially, the drive biasing member may be arranged to move the drive member in an axial direction as soon as the medicament delivery device is activated. If the medicament container is a syringe, the drive member acts to move the plunger of the syringe. The drive biasing member is a preferably a pre-compressed drive spring.

Especially, the feedback spring can be a separate element mounted relative to the drive member. The feedback spring may be accommodated, in the pre-tensioned state, by the drive member and thereby moves with the drive member when the drive member is driven, by the drive biasing member, to move in the axial direction towards proximally. The feedback spring moves together with the drive member in its pre-tensioned state.

Especially, the feedback spring may be mounted to expand, when released, in an axial direction, too, back towards distally. The feedback signal may be caused by the distal end of the feedback spring, or a structure distally of this distal end moved by the feedback spring, tapping against a distal end cap of the medicament delivery device or other structure confining the space into which the feedback spring can expand towards distally.

In embodiments, the feedback spring is a helical spring accommodated in the drive member. The drive biasing member, for example a helical spring, may then extend through an interior of the feedback spring. For example, the drive member may comprise a plunger rod portion, wherein the drive biasing member extends in an interior of the plunger rod portion, and the feedback spring extends around the plunger rod portion, for example in a space between the plunger rod portion and an and an outer portion of the drive member. Such a configuration has the advantage of being space-saving, whereby the medicament delivery device can have same dimensions as prior art medicament delivery devices.

It is not excluded, though, that the feedback spring is a structure of the drive member itself, for example a resilient, pre-tensioned portion thereof. Also such resilient, pre-tensioned portion may be only released when the drive member's end position has been reached, i.e., when the medicament has been expelled.

The feedback spring release mechanism is a mechanism that ensures that the feedback spring is kept in its pre-tensioned state prior to and during medicament delivery and is released when the medicament has been expelled. Such release may for example be due to the drive member reaching its end position.

The feedback spring release mechanism for example may comprise a retention structure that when being in a first position impedes an axial movement of the feedback spring relative to the drive member. The retention structure may be brought into a second position, wherein the feedback spring is released by the retention structure being moved from the first position into the second position.

The retention structure may be a structure of the drive member. The movement from the first position to the second position will then be a movement relative to a drive member body.

The movement from the first position into the second position may be impeded by an abutment structure of the housing before the medicament has been expelled. It may be made possible by the drive member being moved to the end position. It may be a movement caused by a resilient force.

Such a movement from the first position of the retention structure into the second position may, according to a group of embodiments, be a movement in a radial direction, outwardly.

For example, the retention structure may belong to a cantilever of the drive member, the cantilever being one-piece with the drive member body. The retention structure may be formed by an end piece of the cantilever. The movement from the first position into the second position may be caused by a resilient force of the cantilever, and/or by the force of the feedback spring itself, for example if a retention surface of the retention structure is slanted and forms a ramp.

In another group of embodiments, the drive member may comprise a pivot member, and the retention structure may be formed by the pivot member. The movement from the first position into the second position is then a pivoting about a pivot axis. It may be impeded by the abutment structure of the housing before the drive member's end position has been reached, and it may be caused, as soon as the end position has been reached, by the spring force of the feedback spring.

Especially in embodiments in which the movement of the retention structure from the first position into the second position is at least partially caused by a resilient force different from the spring force of the feedback spring, the following may hold: The abutment structure may comprise a pocket for accommodating a portion of the retention structure when the drive member is in its initial position prior to delivery of the medicament. The pocket thereby may allow the retention structure to be, as long as the drive member is in the initial position, in a position in which the cantilever or other resilient means is in a state in which it is not pre-tensioned or pre-tensioned to a lesser extent than during the axial movement of the drive member. For example, the retention structure may be in the second position or in a position between the first and the second position when it is allowed to be accommodated by the pocket. Only during medicament delivery, when the drive member is between the initial position and the end position, will then the retention structure be in its first position. By this measure, it can be avoided that the resilient force wears off over time even if the cantilever or other resilient means is of a material that could otherwise be subject to such wear. Besides that, the cap member may comprise axial protrusions at its distal end holding the feedback spring in place in the initial position.

The housing of the medicament delivery device may comprise a housing sleeve, thus a tubular portion. In addition, it may comprise a distal end cap member. In embodiments, the distal end cap member in addition to forming a distal end cap of the housing also comprises an axially running wall portion extending from the distal end cap towards proximally along an inner surface of the housing sleeve. Such axially running wall portion may form the mentioned abutment structure. The axial extension of the axially running wall portion may be such that the retention structure is immediately proximally of the proximal end of the axially running wall portion when the drive member has reached its end position.

In addition to comprising a housing and a drive member, the medicament delivery device may also comprise an activation member. The activation member may activate the medicament delivery. Before activation becomes possible, for example a proximal end cap needs to be removed by the patient. Activation may be done for example by the medicament delivery device being pressed against the patient's skin at the dose delivery site. By the latter, the activation member may be pushed, relative to the housing, towards distally, against a spring force. The activation by the activation member may especially comprise releasing the drive member so that the drive biasing member is allowed to move the drive member towards proximally.

The activation member may for example comprise a needle guard portion shielding the needle of the syringe (when the medicament container is a syringe) before activation and for example also after the medicament delivery device has been removed from the dose delivery site. The element exerting the force against which the activation member is pushed, therefore, in the present text is called ‘needle guard biasing member’. The needle guard biasing member generally will be separate from both, the drive biasing member and the feedback spring. The needle guard biasing member is preferably a spring.

In addition to concerning a medicament delivery device, the present invention also concerns a medicament delivery assembly comprising the medicament delivery device of the kind described in the present text, pre-assembled with a medicament container, for example a syringe.

Generally, all terms used in the claims are to be interpreted according to their ordinary meaning in the technical field, unless explicitly defined otherwise herein. All references to a/an/the element, apparatus, member, component, means, etc. are to be interpreted openly as referring to at least one instance of the element, apparatus, member component, means, etc., unless explicitly stated otherwise.

### BRIEF DESCRIPTION OF THE DRAWINGS

Embodiments of the present disclosure will now be described by way of example only and with reference to the following accompanying drawings. The drawings show:

FIG. 1 a view of an embodiment of a medicament delivery assembly comprising the medicament delivery device;

FIG. 2 the medicament delivery assembly prepared for medicament delivery;

FIG. 3 the configuration of FIG. 2 without the housing sleeve;

FIG. 4 the assembly of FIG. 2 with the activation member displaced towards distally;

FIG. 5 the configuration of FIG. 4 without the housing sleeve;

FIG. 6 the assembly of FIGS. 2 and 4 in longitudinal section, during medicament delivery;

FIG. 7 the assembly of FIGS. 2 and 4 in longitudinal section, at the end of medicament delivery;

FIG. 8 a view of the end cap member;

FIG. 9 a view of the drive member;

FIG. 10 a detail of FIG. 6;

FIG. 11 a detail of FIG. 7;

FIG. 12 a detail of a distal end of a variant of the medicament delivery device of FIGS. 1-11, in longitudinal section;

FIG. 13 partially, a distal end cap member of the embodiment of FIG. 12;

FIG. 14 a detail of an alternative embodiment, shown without the housing sleeve and in section; and

FIG. 15 a schematic drawing illustrating the working principle of the embodiment of FIG. 14.

### DETAILED DESCRIPTION

The medicament delivery assembly 1 shown in FIG. 1 comprises a medicament delivery device and a syringe 3. The medicament delivery device comprises a housing, encasing, together with a removable proximal end cap 4, the syringe 3. In the depicted embodiment, the housing comprises a housing sleeve 2 and a distal end cap member 5.

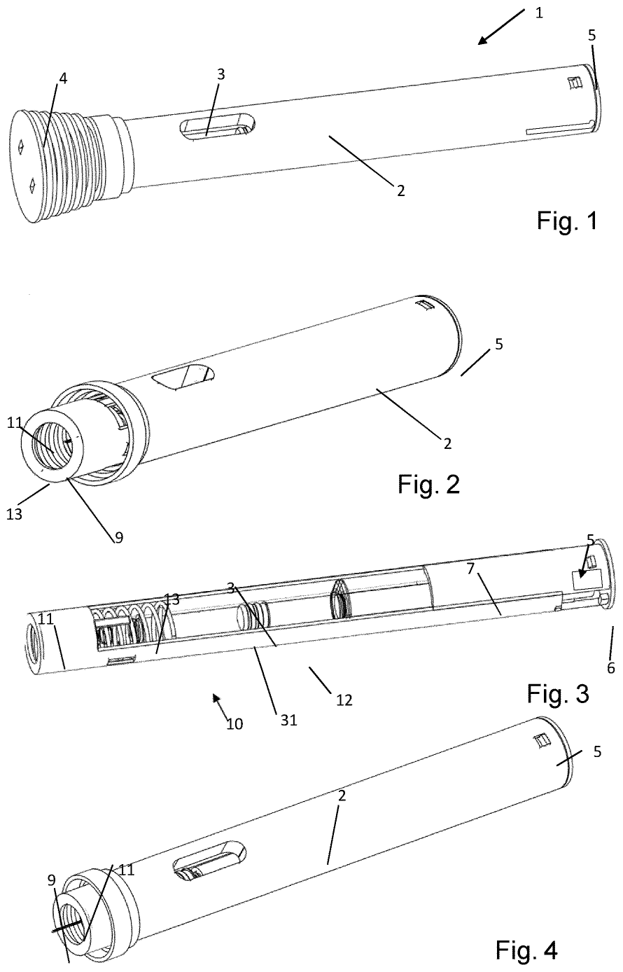

FIG. 2 shows the medicament delivery assembly with a proximal end cap of the medicament delivery device 1 and a needle shield of the syringe 3 (not shown in the figures) removed, ready for medicament delivery by injection. A needle 8 of the syringe is protected by a needle guard portion 11 of an activation member 10.

As can be seen in FIG. 3, depicting the configuration of FIG. 2 without the housing sleeve 2, the activation member 10 has an activation portion 12 extending towards distally from the needle guard portion 11.

For activation, the patient presses the medicament delivery assembly of FIGS. 2 and 3, thus with the proximal end cap and, if applicable, the needle shield, removed, against the dose delivery site and thereby displaces the activation member 10 towards distally relative to the housing, against a force delivered by a needle guard biasing member 13, here a needle guard spring. This results in the configuration of FIG. 4. The activation portion 12 will, by this movement, release the drive member and thereby activate the medicament delivery. There are several possible mechanisms by which a movement of an activation member relative to the housing can activate the medicament delivery. Since the present invention does not depend on the nature of this activation, it is not described in any more detail here.

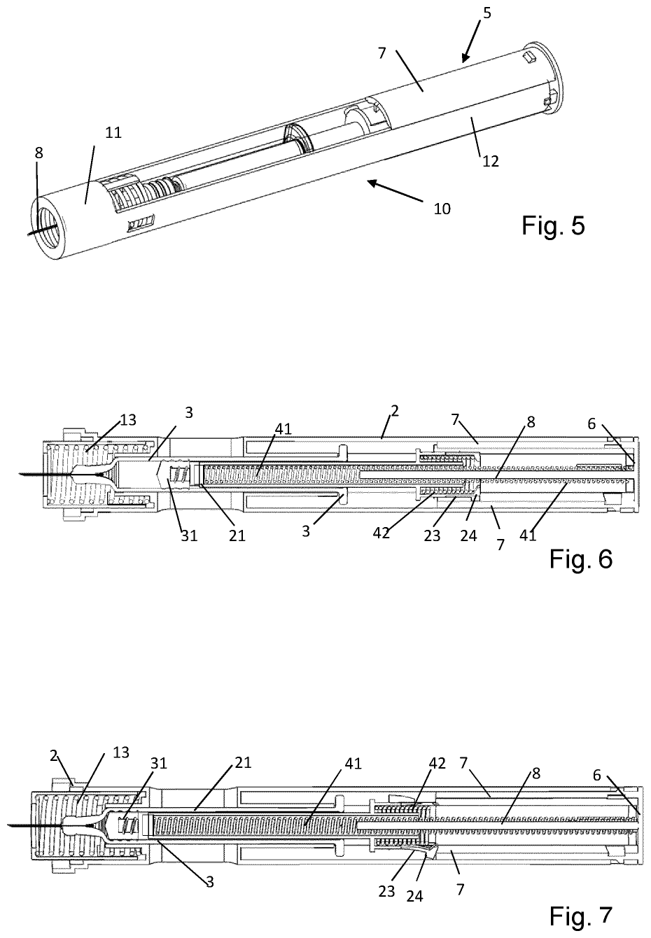

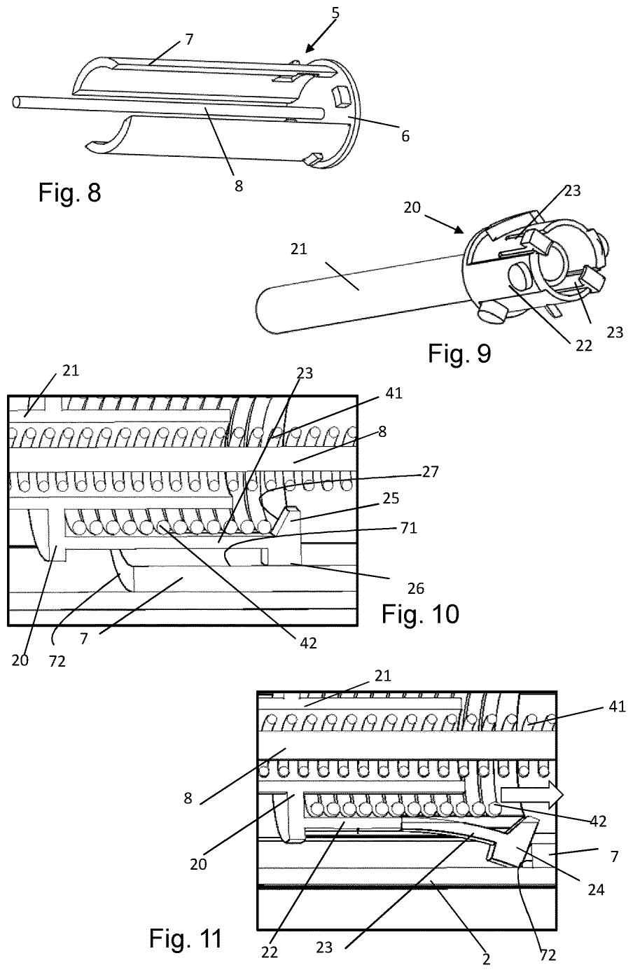

FIG. 5 the configuration of FIG. 4, again without the housing sleeve 2, during medicament delivery. FIGS. 6 and 7 show the assembly of FIG. 4 in longitudinal section, during medicament delivery and at the end of the medicament delivery process, respectively. FIG. 8 shows the distal end cap member 5 of the housing, and FIG. 9 depicts a drive member of the medicament delivery device.

The distal end cap member 5 has, in addition to a distal end cap portion 6, an axially running wall portion 7 and a spring guide portion 8. The spring guide portion 8 guides a drive biasing member 41, here a drive spring, that acts on the drive member 20 to move it in an axial direction, towards proximally, for delivery of the medicament. To this end, the drive member has a plunger rod portion 21 that presses the plunger 31 of the syringe 3 towards proximally when the drive member is moved, by the force of the spring force of the drive spring 41. The plunger rod portion 21 in the shown embodiment is hollow and accommodates a proximal portion of the drive spring 41 that presses against a proximal end bottom of the plunger rod portion 21.

The drive member 20 also has an outer portion 22 that accommodates a feedback spring 42 in a pre-tensioned state. Depending on the activation mechanism, the outer portion 22 may also comprise structures for retention of the drive member in a distal position before delivery and/or for cooperating with the activation portion for activation. In the depicted embodiment, the outer portion 22 further has a plurality of cantilevers 23 with end pieces 24.

The mechanism that releases the feedback spring at the end of the medicament delivery process illustrated in to FIGS. 6 and 7 as well as in FIGS. 10 and 11 showing a detail of FIG. 6 and FIG. 7, respectively.

Before and during delivery of the medicament, each cantilever 23 is prevented from flexing radially outwardly by an outer end piece portion 26 of its end piece 24 abutting against an inner surface 71 of one of the axially running wall portions 7, as can for example be seen in FIG. 10. In this position, the inner end piece portion 25 of the cantilever's end piece 24 prevents the feedback spring 42 contained between the plunger rod portion 21 and the outer portion 21 of the drive member 20 from escaping and thereby keeps it in the pre-tensioned state.

When the end position is reached (FIGS. 7 and 11), the end portion 25 is in a position immediately proximally of a proximal end face 72 of the axially running wall portion 7 and thereby can flex out. This releases the feedback spring 42, as illustrated by the arrow in FIG. 11. The feedback spring 42 as a consequence will expand, and its distal end will tap against the distal end cap 6, giving the patient an audible and possibly also tactile feedback.

Optionally a dedicated, for example ring-shaped feedback element (not shown) of a suitable material may be mounted in contact with the distal end of the spring, and for example connected thereto, whereby it is such feedback element that, driven by the spring, taps against the distal end cap.

The flexing radially outwardly of the cantilever 23 may be due to an elastic force of the cantilever 23 itself, i.e. the cantilever 23 may act as cantilever spring in being pre-tensioned to flex outwardly. In addition or as an alternative, the feedback spring 42 itself may flex the cantilever 23 outwardly or assist such outward movement, in that a retention surface 27 of the inner end piece portion 25 is not parallel to radial directions but slanted to form a ramp, as illustrated in the figures.

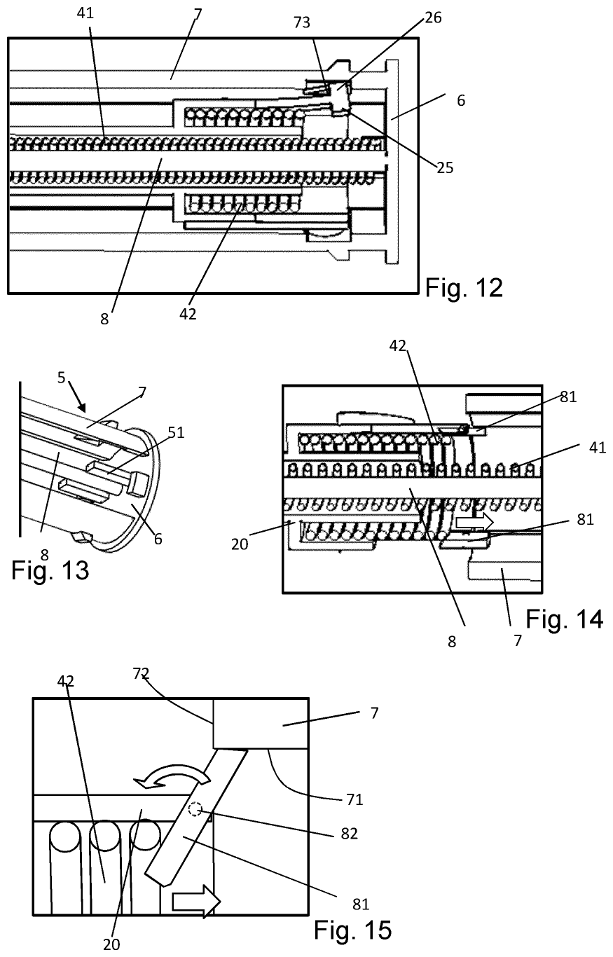

FIG. 12 shows a detail of a distal end of a variant of the medicament delivery device of FIGS. 1-11, in the state before delivery of the medicament. As illustrated, the axially running wall portion has a pocket 73 with a slanted surface. The pocket 73 accommodates the outer end piece portion 26 of the cantilever's end piece before the medicament delivery. For preventing the spring from escaping in this configuration, prior to the medicament delivery, the medicament delivery device may comprise a dedicated structure, such as the axial protrusions 51 of the distal end cap member 5 shown in FIG. 13. The fact that the cantilever can be in the outwardly flexed position before medicament delivery has the advantage that there is no risk of failure of the feedback spring release mechanism due to fatigue. Depending on the material of the drive member, the resilience of the cantilever may otherwise suffer over time if the cantilever is stored the not outwardly flexed position (FIGS. 6 and 10).

FIGS. 14 and 15 yet show details of an alternative embodiment. Instead of comprising a cantilever the drive member comprises a pivot member 81. As illustrated schematically in FIG. 15, the pivot member is pivoting about an axis 82. The feedback spring 42 presses a radially-inner portion of the pivot member 81 towards distally and thereby exerts a torque on the pivot member. Prior to the medicament delivery and during this process, the pivot member is prevented from rotating, due to this torque, by a radially-outer portion abutting against the inner surface 71 of the axially running wall portion, as illustrated in FIG. 15. As soon as the end position is reached, the radially-outer portion gets to a position proximally of the proximal end face 71 and thus is allowed to rotate, as illustrated by the upper arrow in FIG. 15. As a consequence, the pivot member 81 gets into the orientation shown in FIG. 14, and the feedback spring 42 is released as illustrated by the lower arrow in FIG. 15, providing the patient with the feedback signal.

As known from the prior art after the medicament delivery has finished, the patient can remove the medicament delivery assembly, and the needle guard spring 13 acts to move the activation member 8 towards proximally in a position in which the needle guard portion 12 shields the needle. Optionally, a suitable mechanism may lock the activation member in this position.

In the depicted embodiments, the axially running wall portions 7 as well as the spring guide portion 8 belong to the distal end cap member. This is not necessary. Especially, the axially running wall portions 7 may, as an alternative, belong to the housing sleeve 2, for example as flat inward protrusions thereof. As an even further alternative, the housing may be one-piece, with the distal end cap member being integral with the housing sleeve.

Further, in the depicted embodiment, the outer portion and, if present, the cantilever(s) of the drive member are integral with the plunger rod portion. This is not necessary. It would, as an alternative be possible, to compose the drive member of several parts.

The shown springs with the exception of the cantilevers, thus the feedback spring, the drive spring, and the needle guard spring, are all illustrated to be helical springs. Helical springs have the advantage of being capable of providing a long travel while using only a limited space. However, also other kinds springs could be used for the drive spring, and/or the needle guard spring.

The delivery devices described herein can be used for the treatment and/or prophylaxis of one or more of many different types of disorders.

Exemplary disorders include, but are not limited to: rheumatoid arthritis, inflammatory bowel diseases (e.g. Crohn's disease and ulcerative colitis), hypercholesterolaemia and/or dyslipidemia, cardiovascular disease, diabetes (e.g. type 1 or 2 diabetes), psoriasis, psoriatic arthritis, spondyloarthritis, hidradenitis suppurativa, Sjögren's syndrome, migraine, cluster headache, multiple sclerosis, neuromyelitis optica spectrum disorder, anaemia, thalassemia, paroxysmal nocturnal hemoglobinuria, hemolytic anaemia, hereditary angioedema, systemic lupus erythematosus, lupus nephritis, myasthenia gravis, Behçet's disease, hemophagocytic lymphohistiocytosis, atopic dermatitis, retinal diseases (e.g., age-related macular degeneration, diabetic macular edema), uveitis, infectious diseases, bone diseases (e.g., osteoporosis, osteopenia), asthma, chronic obstructive pulmonary disease, thyroid eye disease, nasal polyps, transplant, acute hypoglycaemia, obesity, anaphylaxis, allergies, sickle cell disease, Alzheimer's disease, Parkinson's disease, dementia with Lewy bodies, systemic infusion reactions, immunoglobulin E (IgE)-mediated hypersensitivity reactions, cytokine release syndrome, immune deficiencies (e.g., primary immunodeficiency, chronic inflammatory demyelinating polyneuropathy), enzyme deficiencies (e.g., Pompe disease, Fabry disease, Gaucher disease), growth factor deficiencies, hormone deficiencies, coagulation disorders (e.g., hemophilia, von Willebrand disease, Factor V Leiden), and cancer.

Exemplary types of drugs that could be included in the delivery devices described herein include, but are not limited to, small molecules, hormones, cytokines, blood products, enzymes, vaccines, anticoagulants, immunosuppressants, antibodies, antibody-drug conjugates, neutralizing antibodies, reversal agents, radioligand therapies, radioisotopes and/or nuclear medicines, diagnostic agents, bispecific antibodies, proteins, fusion proteins, peptibodies, polypeptides, pegylated proteins, protein fragments, nucleotides, protein analogues, protein variants, protein precursors, protein derivatives, chimeric antigen receptor T cell therapies, cell or gene therapies, oncolytic viruses, or immunotherapies.

Exemplary drugs that could be included in the delivery devices described herein include, but are not limited to, immuno-oncology or bio-oncology medications such as immune checkpoints, cytokines, chemokines, clusters of differentiation, interleukins, integrins, growth factors, coagulation factors, enzymes, enzyme inhibitors, retinoids, steroids, signaling proteins, pro-apoptotic proteins, anti-apoptotic proteins, T-cell receptors, B-cell receptors, or costimulatory proteins.

Exemplary drugs that could be included in the delivery devices described herein include, but are not limited to, those exhibiting a proposed mechanism of action, such as human epidermal growth factor receptor 2 (HER-2) receptor modulators, interleukin (IL) modulators, interferon (IFN) modulators, complement modulators, glucagon-like peptide-1 (GLP-1) modulators, glucose-dependent insulinotropic polypeptide (GIP) modulators, cluster of differentiation 38 (CD38) modulators, cluster of differentiation 22 (CD22) modulators, C1 esterase modulators, bradykinin modulators, C—C chemokine receptor type 4 (CCR4) modulators, vascular endothelial growth factor (VEGF) modulators, B-cell activating factor (BAFF), P-selectin modulators, neonatal Fc receptor (FcRn) modulators, calcitonin gene-related peptide (CGRP) modulators, epidermal growth factor receptor (EGFR) modulators, cluster of differentiation 79B (CD79B) modulators, tumor-associated calcium signal transducer 2 (Trop-2) modulators, cluster of differentiation 52 (CD52) modulators, B-cell maturation antigen (BCMA) modulators, enzyme modulators, platelet-derived growth factor receptor A (PDGFRA) modulators, cluster of differentiation 319 (CD319 or SLAMF7) modulators, programmed cell death protein 1 and programmed death-ligand 1 (PD-1/PD-L1) inhibitors/modulators, B-lymphocyte antigen cluster of differentiation 19 (CD19) inhibitors, B-lymphocyte antigen cluster of differentiation 20 (CD20) modulators, cluster of differentiation 3 (CD3) modulators, cytotoxic T-lymphocyte-associated protein 4 (CTLA-4) inhibitors, T-cell immunoglobulin and mucin-domain containing-3 (TIM-3) modulators, T cell immunoreceptor with Ig and ITIM domains (TIGIT) modulators, V-domain Ig suppressor of T cell activation (VISTA) modulators, indoleamine 2,3-dioxygenase (IDO or INDO) modulators, poliovirus receptor-related immunoglobulin domain-containing protein (PVRIG) modulators, lymphocyte-activation gene 3 (LAG3; also known as cluster of differentiation 223 or CD223) antagonists, cluster of differentiation 276 (CD276 or B7-H3) antigen modulators, cluster of differentiation 47 (CD47) antagonists, cluster of differentiation 30 (CD30) modulators, cluster of differentiation 73 (CD73) modulators, cluster of differentiation 66 (CD66) modulators, cluster of differentiation w137 (CDw137) agonists, cluster of differentiation 158 (CD158) modulators, cluster of differentiation 27 (CD27) modulators, cluster of differentiation 58 (CD58) modulators, cluster of differentiation 80 (CD80) modulators, cluster of differentiation 33 (CD33) modulators, cluster of differentiation 159 (CD159 or NKG2) modulators, glucocorticoid-induced TNFR-related (GITR) protein modulators, Killer Ig-like receptor (KIR) modulators, growth arrest-specific protein 6 (GAS6)/AXL pathway modulators, A proliferation-inducing ligand (APRIL) receptor modulators, human leukocyte antigen (HLA) modulators, epidermal growth factor receptor (EGFR) modulators, B-lymphocyte cell adhesion molecule modulators, cluster of differentiation w123 (CDw123) modulators, Erbb2 tyrosine kinase receptor modulators, endoglin modulators, mucin modulators, mesothelin modulators, hepatitis A virus cellular receptor 2 (HAVCR2) antagonists, cancer-testis antigen (CTA) modulators, tumor necrosis factor receptor superfamily, member 4 (TNFRSF4 or OX40) modulators, adenosine receptor modulators, inducible T cell co-stimulator (ICOS) modulators, cluster of differentiation 40 (CD40) modulators, tumor-infiltrating lymphocytes (TIL) therapies, or T-cell receptor (TCR) therapies.

Exemplary drugs that could be included in the delivery devices described herein include, but are not limited to: etanercept, abatacept, adalimumab, evolocumab, exenatide, secukinumab, erenumab, galcanezumab, fremanezumab-vfrm, alirocumab, methotrexate (amethopterin), tocilizumab, interferon beta-1a, interferon beta-1b, peginterferon beta-1a, sumatriptan, darbepoetin alfa, belimumab, sarilumab, semaglutide, dupilumab, reslizumab, omalizumab, glucagon, epinephrine, naloxone, insulin, amylin, vedolizumab, eculizumab, ravulizumab, crizanlizumab-tmca, certolizumab pegol, satralizumab, denosumab, romosozumab, benralizumab, emicizumab, tildrakizumab, ocrelizumab, ofatumumab, natalizumab, mepolizumab, risankizumab-rzaa, ixekizumab, and immune globulins.

Exemplary drugs that could be included in the delivery devices described herein may also include, but are not limited to, oncology treatments such as ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab.

Exemplary drugs that could be included in the delivery devices described herein include “generic” or biosimilar equivalents of any of the foregoing, and the foregoing molecular names should not be construed as limiting to the “innovator” or “branded” version of each, as in the non-limiting example of innovator medicament adalimumab and biosimilars such as adalimumab-afzb, adalimumab-atto, adalimumab-adbm, and adalimumab-adaz.

Exemplary drugs that could be included in the delivery devices described herein also include, but are not limited to, those used for adjuvant or neoadjuvant chemotherapy, such as an alkylating agent, plant alkaloid, antitumor antibiotic, antimetabolite, or topoisomerase inhibitor, enzyme, retinoid, or corticosteroid. Exemplary chemotherapy drugs include, by way of example but not limitation, 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine.

Exemplary drugs that could be included in the delivery devices described herein also include, but are not limited to, analgesics (e.g., acetaminophen), antipyretics, corticosteroids (e.g. hydrocortisone, dexamethasone, or methylprednisolone), antihistamines (e.g., diphenhydramine or famotidine), antiemetics (e.g., ondansetron), antibiotics, antiseptics, anticoagulants, fibrinolytics (e.g., recombinant tissue plasminogen activator [r-TPA]), antithrombolytics, or diluents such as sterile water for injection (SWFI), 0.9% Normal Saline, 0.45% normal saline, 5% dextrose in water, 5% dextrose in 0.45% normal saline, Lactated Ringer's solution, Heparin Lock Flush solution, 100 U/mL Heparin Lock Flush Solution, or 5000 U/mL Heparin Lock Flush Solution.

Pharmaceutical formulations including, but not limited to, any drug described herein are also contemplated for use in the delivery devices described herein, for example pharmaceutical formulations comprising a drug as listed herein (or a pharmaceutically acceptable salt of the drug) and a pharmaceutically acceptable carrier. Such formulations may include one or more other active ingredients (e.g., as a combination of one or more active drugs), or may be the only active ingredient present, and may also include separately administered or co-formulated dispersion enhancers (e.g. an animal-derived, human-derived, or recombinant hyaluronidase enzyme), concentration modifiers or enhancers, stabilizers, buffers, or other excipients.

Exemplary drugs that could be included in the delivery devices described herein include, but are not limited to, a multi-medication treatment regimen such as AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, Mini-CHOP, Maxi-CHOP, VR-CAP, Dose-Dense CHOP, EPOCH, Dose-Adjusted EPOCH, R-EPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOPAD, 7+3, 5+2, 7+4, MEC, CVP, RBAC500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DT-PACE, VTD-PACE, DCEP, ATG, VAC, VeIP, OFF, GTX, CAV, AD, MAID, AIM, VAC-IE, ADOC, or PE. Various modifications to the embodiments described are possible and will occur to those skilled in the art without departing from the invention which is defined by the following claims.

1-14. (canceled)

15. A medicament delivery device for accommodating a medicament container, the medicament delivery device comprising:
a drive mechanism equipped to act on the medicament container for expelling a medicament therefrom;
a feedback spring in a pre-tensioned state, said feedback spring being an axial helical spring; and
a feedback spring release mechanism equipped to release the feedback spring from the pre-tensioned state when the medicament has been expelled, whereby release of the feedback spring causes a feedback signal.

16. The medicament delivery device according to claim 15, further comprising a housing, wherein the drive mechanism comprises a pre-loaded drive biasing member and a drive member arranged to be moved, by the drive biasing member, relative to the housing in an axial direction, axial referring to a longitudinal axis of the medicament delivery device.

17. The medicament delivery device according to claim 16, wherein the feedback spring is accommodated, in the pre-tensioned state, by the drive member and moves with the drive member when the drive member is driven by the drive biasing member, the feedback spring moving together with the drive member in its pre-tensioned state.

18. The medicament delivery device according to claim 16, wherein the drive member comprises a plunger rod portion for moving the plunger of a syringe as the medicament container.

19. The medicament delivery device according to claim 16, wherein the drive biasing member is a helical spring, wherein the feedback spring is a helical spring, and wherein the drive biasing member has a smaller diameter than the feedback spring and extends through an interior of the feedback spring.

20. The medicament delivery device according to claim 16, wherein the feedback spring release mechanism comprises a retention structure of the drive member, wherein retention structure impedes an axial movement of the feedback spring relative to the drive member when it is in a first position, wherein the retention structure is movable, to a second position, and wherein the feedback spring is released by the retention structure being moved from the first position into the second position.

21. The medicament delivery device according to claim 20, wherein a movement from the first position into the second position is an outwardly movement in a radial direction, and wherein the retention structure is moved from the first position into the second position by a resilient force.

22. The medicament delivery device according to claim 21, wherein the retention structure belongs to a cantilever of the drive member, the cantilever being one-piece with a drive member body of the drive member.

23. The medicament delivery device according to claim 20, wherein the retention structure belongs to a pivot member, the pivot member pivoting from the first position to the second position by a force of the feedback spring.

24. The medicament delivery device according to claim 20, wherein the retention structure is prevented, by an abutment structure of the housing, from being moved from the first position into the second position while the drive member is moved in the axial direction before it has reached an end position, and is allowed to be moved from the first position into the second position when the drive member has reached the end position.

25. The medicament delivery device according to claim 24, wherein the housing comprises a pocket in the abutment structure, wherein the retention structure reaches into the pocket when the drive member is in an initial position prior to the expelling of the medicament, the cap member comprising axial protrusions at its distal end holding the feedback spring in place in the initial position.

26. The medicament delivery device according to claim 24, wherein the housing comprises a housing sleeve and a distal end cap member, and wherein the abutment structure is an axially running wall portion of the distal end cap member, the axially running wall portion extending towards proximally from a distal end cap portion of the distal end cap member.

27. The medicament delivery device according to claim 16, further comprising an activation member and a needle guard biasing member, wherein the activation member is movable relative to the housing against a force delivered by the needle guard biasing member, to release the drive biasing member and to thereby activate the movement of the drive member relative to the housing.

28. A medicament delivery assembly, comprising the medicament delivery device according to claim 15, and further comprising the medicament container assembled with the medicament delivery device.
